(19) United States
(12) Patent Application Publication
(10) Pub. No.: US 2023/0313238 A1
(43) Pub. Date: Oct. 5, 2023

(54) METHOD OF PRODUCING GAMMA-BUTYROLACTONE FROM BIOMASS

(71) Applicant: CJ CHEILJEDANG CORPORATION, Jung-gu (KR)

(72) Inventors: John LICATA, Groveland, MA (US); Derek SAMUELSON, Woburn, MA (US); Harvey Hayes MORGAN, III, Fort Mill, SC (US)

(73) Assignee: CJ CHEILJEDANG CORPORATION, Jung-gu (KR)

(21) Appl. No.: 18/191,716

(22) Filed: Mar. 28, 2023

Related U.S. Application Data
(60) Provisional application No. 63/325,243, filed on Mar. 30, 2022.

(51) Int. Cl. C12P 7/38

(52) U.S. Cl. CPC C12P 7/38

(57) ABSTRACT

An improved method of producing gamma-butyrolactone (GBL) product with high purity in high yield from a starting biomass containing poly-4-hydroxybutyrate (P4HB) and water is disclosed. The method includes recycling a part of GBL product to replace the water to obtain a substantially water-free biomass slurry that is subjected to a conversion process to convert the P4HB to GBL.

### CROSS REFERENCE TO RELATED APPLICATIONS

This application claims benefit to U.S. Provisional Application No. 63/325,243 filed Mar. 30, 2022, the content of which is incorporated herein by reference in its entirety.

### FIELD OF THE DISCLOSURE

The present disclosure relates to an improved process for producing gamma-butyrolactone (GBL) from biomass.

### BACKGROUND OF THE DISCLOSURE

With dwindling petroleum resources, increasing energy prices, and environmental concerns, development of energy efficient biorefinery processes to produce bio-based chemicals from renewable, low cost, carbon resources offers a unique solution to overcoming the increasing limitations of petroleum-based chemicals.

One chemical with wide industrial and pharmaceutical uses that could be manufactured using a biorefinery process is gamma-butyrolactone (GBL). The global market demand for GBL has been estimated at 850 million lbs/yr, translating to total sales of $1 billion annually. GBL is a colorless, weak odor liquid that is used predominantly as an intermediate in the manufacture of commercially important chemicals such as 1,4-butanediol (BDO), tetrahydrofuran (THF), N-methylpyrrolidone (NMP), N-ethylpyrrolidone (NEP), 2-pyrrolidinone, N-vinylpyrrolidone (NVP), polyvinylpyrrolidone (PVP) and so forth. These chemicals have applications in high performance solvents for electronics, lube oil extraction, magnetic wire coatings, engineering resins, pharmaceutical intermediates, cosmetics, hair spray and high valued polymers. GBL by itself has many uses including as a solvent for paint stripping, degreaser, viscosity modifier for polyurethanes, dispersant for water soluble inks, curing agent for urethanes and polyamides, etchant for metal coated plastics, rubber additive and herbicide ingredient.

Petroleum-based GBL is manufactured by several different chemical processes. For example, GBL is synthesized by dehydration of gamma-hydroxybutyric acid (GHB), by the reaction of acetylene with formaldehyde or vapor phase hydrogenation of maleic anhydride or succinic anhydride and their esters. The latter two methods are respectively known as the Reppe process and the Davy process. The Reppe process was developed in the 1940's and historically was the first commercial route to making 1,4-butanediol. The process starts by reacting acetylene and formaldehyde together which is then followed by a series of hydrogenation stages to obtain BDO and finally dehydrogenation to generate GBL. The main disadvantages of this process are that the starting reactants are quite hazardous and generally present the manufacturer with handling and environmental challenges. Additionally, acetylene is a relatively expensive starting material.

The Davy Process, developed in the 1990's, uses a multistage process that starts by reacting molten maleic anhydride with methanol to produce monomethyl maleate. Next the monomethyl maleate is converted from mono to dimethyl maleate in the presence of an acid resin catalyst. Using catalytic vapor phase hydrogenation, the dimethyl maleate is converted to dimethyl succinate and then finally through a series of additional reactions to a GBL. The final product is refined to obtain the high purity GBL. Many patents describe the various types of hydrogenation catalysts used to convert maleic anhydride or succinic anhydride to GBL. These include copper chromite (described in U.S. Pat. No. 3,065,243), copper chromite with nickel (U.S. Pat. No. 4,006,165), and mixtures of copper, zinc or aluminum oxides (U.S. Pat. No. 5,347,021) as well as reduced copper and aluminum oxides mixtures (U.S. Pat. No. 6,075,153).

Even with the large number of available hydrogenation catalysts for GBL production, there are deficiencies which need to be overcome such as yield, selectivity, ease of product recovery and cost.

U.S. Pat. No. 9,084,467 discloses a process for production of a bio-based gamma-butyrolactone product, comprising a) combining a genetically engineered biomass comprising poly-4-hydroxybutyrate (P4HB) and a catalyst; and b) heating the biomass with the catalyst to convert the P4HB to a GBL product, wherein the catalyst is sodium carbonate or calcium hydroxide.

U.S. Patent Application Publication No. 2014-0170714 discloses a process for production and purification of a biobased GBL, comprising a) combining a genetically engineered biomass comprising poly-4-hydroxybutyrate (P4HB) and a catalyst; b) heating the biomass with the catalyst to convert the P4HB to a GBL product; and c) removing impurities from the GBL product forming a pure GBL.

According to U.S. Pat. No. 9,084,467 and US Patent Application Publication No. 2014-0170714, during or following production (e.g., culturing) of the P4HB biomass, the biomass is combined with a catalyst under suitable conditions to help convert the P4HB polymer to high purity GBL product. The catalyst (in solid or solution form) and biomass are combined for example by mixing, flocculation, centrifuging or spray drying, or other suitable method known in the art for promoting the interaction of the biomass and catalyst driving an efficient and specific conversion of P4HB to GBL. The biomass is initially dried, for example at a temperature between about 100° C. and about 150° C. and for an amount of time to reduce the water content of the biomass. The dried biomass is then re-suspended in water prior to combining with the catalyst to proceed with the conversion to obtain GBL. The entire contents of U.S. Pat. No. 9,084,467 and US Patent Application Publication No. 2014-0170714 are incorporated by reference.

The conventional process for making GBL from poly (4-hydroxybutyric acid) (P4HB), such as the process in U.S. Pat. No. 9,084,467 and U.S. Patent Application Publication No. 2014-0170714 includes spray drying (removing water) of biomass from fermentation broth or drying using, e.g., a hot drum drier (hot metal surface of e.g., 300° C.), followed by pyrolysis, i.e., a reaction with a catalyst to convert P4HB to GBL. The conventional drying process of the biomass is energy intensive and inefficient. Drying is necessary because the biomass containing P4HB needs to be heated to above 150° C. for converting P4HB to GBL. The resulting GBL is very crude with significant amount of N-methylpyrrolidone (NMP) and a number of other organic compounds (furfurols and amines) which can impart color and odor to the product requiring post treatment and distillation with high reflux rate and significant number of trays in the column. The conversion process itself can require high surface temperatures and some difficult solids handling challenges.

A need therefore exists to develop new GBL manufacturing processes that address not only improvements in the yield, purity, and cost of the product but also use sustainable starting materials that have a more positive impact on the environment.

### SUMMARY OF THE DISCLOSURE

An aspect of the present disclosure is directed to processes for producing high purity, high yield, biobased, gamma-butyrolactone (GBL) product from biobased renewable carbon resources, which comprises removing water from the biobased renewable carbon resources (starting biomass) and introducing a recycle stream of GBL to replace at least part of the removed water. In one aspect, a process for the production of gamma-butyrolactone (GBL) product from a starting biomass containing poly-4-hydroxybutyrate (P4HB), comprises removing water from the starting biomass by replacing the water with a recycle stream of vapor or liquid GBL to obtain a substantially water-free biomass in a suspension or solution form comprising the P4HB dissolved or suspended in the GBL, and combining thus-obtained substantially water-free biomass comprising P4HB and GBL with a catalyst, heating the biomass containing P4HB with the catalyst, to convert P4HB to the gamma-butyrolactone (GBL) product. Therefore, the vapor or liquid GBL introduced to the biomass, in particular, the vapor or liquid recycled GBL serves as a solvent for P4HB (extracted P4HB from host cells in the biomass). The term “substantially water-free” or “substantially free of water” as used throughout the disclosure is intended to refer that the biomass that is introduced to a conversion reaction (i.e., combining with catalyst) contains about 5 wt % or less, about 4.5 wt % or less, about 4 wt % or less, about 3.5 wt % or less, about 3 wt % or less, about 2.5 wt % or less, about 2 wt % or less, about 2.5 wt % or less, about 2 wt % or less, about 1.5 wt % or less, or about 1 wt % or less, based on the weight of the biomass that is combined with a conversion reaction catalyst. In some embodiments, biomass that is combined with a conversion reaction catalyst does not include any added water. In a non-limiting embodiment, the process can be carried out using a multiple-effect evaporator.

A multiple-effect evaporator is an apparatus for efficiently using the heat from steam to evaporate water. Water is boiled in a sequence of vessels (columns), each held at a lower pressure than the last. Because the boiling temperature of water decreases as pressure decreases, the vapor boiled off in one vessel can be used to heat the next, and only the first vessel (at the highest pressure) requires an external source of heat. While in theory, evaporators may be built with an arbitrarily large number of stages, evaporators with more than four stages are rarely practical except in systems where the liquor is the desired product such as in chemical recovery systems where up to seven effects are used.

The process of the present disclosure removes and replaces water contained in the starting biomass at least one time, at least two times, or at least three times with liquid or vapor GBL that serves as a solvent for P4HB, to obtain a suspension or solution of biomass that is to be combined with a conversion reaction catalyst. The suspension or solution of biomass contains P4HB (extracted from host cells in the biomass) dissolved or suspended in GBL that replaced water contained in the starting biomass. Starting biomass from fermentation broth generally contains about 10-30% biomass (solid) and the remainder is water. The entire process from the initial loading of the starting biomass to the completion of the conversion reaction is fluid without using an additional solvent such as added water (because GBL functions/serves as a solvent for P4HB) and is without drying process. The use of liquid or vapor GBL that is recycled back to the biomass as a solvent improves the purity and yield of the GBL obtained as a conversion product, and increases energy efficiency of the process.

Introducing a recycle vapor or liquid stream of GBL to the biomass to obtain a substantially water-free biomass suspension or solution, that is to be combined with a catalyst for converting P4HB to GBL, can help lower the conversion reaction temperature and reduce the amount of high boiling foul smelling impurities. This method greatly improves heat transfer and reduces the amount of energy used in the process by at least 50%.

The content of P4HB in the starting biomass is greater than 10% by weight of the total starting biomass. The starting biomass or biobased renewable starting materials can be genetically engineered biomass as described in U.S. Pat No. 9,084,467 B2, the entire content of which is incorporated herein by reference. The advantages of this bioprocess are that it uses a renewable carbon source as the feedstock material, the genetically engineered microbe produces P4HB in very high yield without adverse toxicity effects to the host cell (which could limit process efficiency) and, when combined with a catalyst and heated, is capable of producing biobased GBL in good yield with good purity.

In some embodiments, the vapor or liquid GBL introduced to replace the water contained in the starting biomass is entirely recycled stream (liquid or vapor) GBL that is produced from the P4HB by the conversion process. In some other embodiments, a part of the vapor or liquid GBL introduced to replace the water contained in the starting biomass is added GBL and the remainder of the vapor or liquid GBL is recycled stream. The added GBL may be biobased GBL or petroleum-based GBL. The process according to the disclosure may be a continuous process and the added GBL may be introduced only at the initial stage of the process until the amount of recycled GBL can replace the substantially all of the water in the biomass, and the entirety of the GBL introduced into the biomass can be recycle GBL. The term “added GBL” is intended to refer to GBL introduced to the biomass from a source other than recycled GBL. The disclosure also pertains to a biobased gamma-butyrolactone (GBL) product produced by the processes described herein. In certain aspects, the amount of GBL in the conversion product produced (prior to post-production purification such as distillation) is 85 wt %, or greater than 85 wt %, or greater than 90 wt %, or greater than 91 wt %, or greater than 92 wt %, or greater than 93 wt %, or greater than 94 wt %, or greater than 95 wt %. In a further aspect, the starting biomass comprising poly-4-hydroxybutyrate produced from renewable resources which is suitable as a feedstock for producing gamma-butyrolactone product, contains poly-4-hydroxybutyrate greater than 10%, greater than 20%, greater than 30%, greater than 40%, or greater than 50% by weight of the biomass.

Specifically, the present disclosure relates to the following items.

Item 1. A method of producing gamma-butyrolactone (GBL) product from a starting biomass containing water and poly-4-hydroxybutyrate (P4HB)-containing cells, the method comprising:
(a) introducing the starting biomass to an evaporator,
(b) introducing liquid or vapor GBL to the evaporator as a solvent in a weight equivalent to or less than a weight of water removed to obtain a biomass suspension or solution that is substantially free of water and comprises P4HB dissolved or suspended in the GBL,
(c) combining the biomass suspension or solution that is substantially free of water and a conversion catalyst and heating resulting mixture to convert the P4HB to GBL, and
(d) collecting the GBL,
wherein the liquid or vapor GBL introduced in step (b) is a recycle GBL that is a part of the collected GBL obtained in step (d) and recycled back to the evaporator to be mixed as a solvent with the biomass, and
wherein the method does not include a drying.

Item 2. The method of item 1, which further comprises (e) mixing, stirring, vortex, or agitation to promote extraction of P4HB from host cells into GBL.

Item 3. The method of item 1, which further comprises, prior to step (c), (f) removing solids from the biomass suspension or solution that is substantially free of water, by filtration, precipitation, or centrifugation.

Item 4. The method of item 1, wherein no water is added in steps (a), (b), and (c).

Item 5. The method of item 1, wherein the evaporator comprises multiple serial evaporators that are in fluid communication with each other and the recycled liquid GBL is introduced to one or more of the evaporators.

Item 6. The method of item 1, wherein the evaporator contains a first evaporator, a second evaporator, and a third evaporator, which are in fluid communication with each other in serial in this order, and the recycled GBL is introduced into the second evaporator and/or the third evaporator.

Item 7. The method of item 1, wherein the step (a) is carried out at a temperature of from about 60° C.-100° C. under vacuum or at atmospheric pressure.

Item 8. The method of item 6, wherein about 20-50% of water contained in the starting biomass is removed in the first evaporator, about 20-45% of water contained in the starting biomass is removed in the second evaporator, and about 5-35% water contained in the starting biomass is removed in the third evaporator; and wherein the recycled GBL is introduced to replace the water removed from the first and the second evaporator.

Item 9. The method of item 1, wherein the biomass solution or suspension that is substantially free of water has a water content of about 5 wt % or less, about 4 wt % or less, about 3.5 wt % or less, about 3 wt % or less, about 2.5 wt % or less, about 2 wt % or less, about 1.5 wt % or less, about 1 wt % or less, based on weight of the biomass solution or suspension.

Item 10. The method of item 1, wherein the step (a) is carried out at a temperature from about 70° C.-90° C. under vacuum.

Item 11. The method of item 1, wherein the step (a) is carried out for a time period from about 5 minutes to about 2 hours.

Item 12. The method of item 1, wherein the collected GBL in step (d) comprises less than 5% by weight of side products.

Item 13. The method of item 1, wherein the conversion of step (c) is carried out in the presence of a catalyst.

Item 14. The method of item 13, wherein the catalyst is sodium carbonate or calcium hydroxide.

Item 15. The method of item 1, wherein a yield of the GBL product is about 85% by weight or greater based on one gram of a GBL in a conversion product per gram of poly-4-hydroxybutyrate.

Item 16. The method of item 1, wherein the starting biomass is a genetically engineered biomass from a host cell that includes a non-naturally occurring amount of P4HB. item 1.

Item 17. A biobased gamma-butyrolactone product produced by the method of

Item 18. The biobased gamma-butyrolactone product of item 17, wherein the gamma-butyrolactone product comprises less than 5% by weight of side products.

### BRIEF DESCRIPTION OF THE DRAWINGS

FIG. 1 is a schematic diagram of one exemplary process for the production of GBL from P4HB biomass.

FIG. 2 shows 30% solution of 300K Da P4HB in GBL (left) (P4HB is soluble in GBL) and extraction of P4HB from biomass with GBL in Rotovap (right).

FIG. 3 is a picture of P4HB extracted using GBL and precipitated into water.

FIG. 4 is a schematic diagram of another exemplary process for the production of GBL from P4HB biomass.

### DETAILED DESCRIPTION OF THE DISCLOSURE

The present disclosure relates to a novel process which introduces a recycle stream of GBL to the reaction step to help lower the reaction temperature and reduce the amount of high boiling foul smelling impurities. The process greatly improves heat transfer and reduces the amount of energy used in the process by at least 50%, compared to an existing process including drying of the biomass.

The processes according to embodiments eliminate drying (spray drying or drum drying) of biomass, and thus saves up to 50% energy cost, compared to an existing process including drying of the biomass.

The processes according to embodiments reduce the peak temperature experienced by the biomass during the conversion reaction where P4HB is converted to GBL by the action of a catalysis under heat treatment, reducing the amount of side reactions that produces a significant amount N-containing compounds, such as N-methylpyrrolidone (NMP). NMP is a known carcinogen and a persistent impurity which is difficult to separate from GBL because the BP (boiling point) delta is only 2° C. Removing N from the biomass prior to the conversion reaction can reduce the amount of NMP impurity.

The processes according to embodiments eliminate the need for any solids handling at the manufacturing plant, thanks to removal of biomass drying step.

The processes according to embodiments are significantly cost-effective, because the conversion product contains a high yield highly pure GBL, the subsequent separation or purification steps employing, for example, distillation/condensation column(s) can be carried out with reduced separation capacity to obtain desired purity and/or yield of final GBL. In some embodiments, the resulting GBL product concentrate is >99% GBL without any distillation. This slurry/solution may be fed to a reactor to further break down the rest of the polymer prior to recovery. The processes according to embodiments are a very clean process virtually eliminating high temperature breakdown byproducts of biomass and potentially can be made 99.9% pure GBL with only one distillation step.

In certain aspects, a genetically engineered P4HB biomass from a host organism serves as a renewable source for converting poly-4-hydroxybutyrate (P4HB) to GBL. In some embodiments, a source of the renewable feedstock is selected from glucose, fructose, sucrose, arabinose, maltose, lactose, xylose, fatty acids, vegetable oils, and biomass derived synthesis gas or a combination of two or more of these. In certain embodiments, the heating of the substantially water-free biomass suspension or solution and catalyst (i.e., the conversion reaction) is at a temperature of between about 190° C. to about 300° C. In some embodiments, the heating temperature is about 195° C. to about 285° C., or about 200° C. to about 275° C., or about 200° C. to about 260° C., or about 200° C. to about 250° C., about 195° C. to about 265° C., or about 200° C. to about 260° C., or about 205° C. to about 250° C., or about 210° C. to about 240° C. In other embodiments, the heating temperature is about 190-220° C. In some embodiments, the multiple effect evaporation process reduces the water content of the biomass to about 15 wt % or less, about 12 wt % or less, about 10 wt % or less, about 9 wt % or less, about 8 wt % or less, about 7 wt % or less, about 6 wt % or less, about 5 wt % or less. In the embodiments described, the heating of the substantially water-free biomass suspension or solution and catalyst is for a time period from about 30 seconds to about 5 minutes, or about 1 minute to about 4 minutes, about 2 minutes to about 3 minutes, or is from about 5 minutes to about 2 hours, or from about 10 minutes to about 90 minutes, or from about 20 minutes to about 80 minutes, or from about 30 minutes to about 70 minutes, or from about 40 minutes to about 60 minutes, or from about 45 minutes to about 55 minutes. In certain embodiments the gamma-butyrolactone comprises less than 10%, or less than 9%, or less than 8%, or less than 7%, or less than 6%, or less than 5%, or less than 4%, or less than 3%, or less than 2%, or less than 1% of undesired side products.

In certain embodiments, the catalyst of the conversion reaction is sodium carbonate or calcium hydroxide. The weight percent of catalyst is in the range of about 4% to about 50%, or about 8% to about 45%, or about 10% to about 40%, or about 15% to about 35%, or about 20% to about 30%, or about 25% to about 30% based on the total weight of the mixture of the substantially water-free biomass suspension or solution and the catalyst. In particular embodiments, the weight % of the catalyst is in the range of about 4% to about 50%, and the heating of the substantially water-free biomass suspension or solution and catalyst is at about 300° C., or about 280° C., or about 250° C., or about 240° C., or about 230° C., or about 220° C., or about 210° C., or about 200° C. In some embodiments, the catalyst is 4%, or 5%, or 6%, or 7%, or 8%, or 9%, or 10%, or 11%, or 12%, or 13%, or 14%, or 15%, by weight calcium hydroxide and the heating is at a temperature of 300° C., or about 290° C., or about 280° C., or about 270° C., or about 260° C., or about 250° C., or about 240° C., or about 230° C., or about 225° C., or about 220° C., or about 215° C., or about 210° C., or about 205° C., or about 200° C., or about 195° C.

For the purposes of this disclosure, P4HB is defined to also include the copolymer of 4-hydroxy butyrate with 3-hydroxy butyrate where the percentage (%) of 4-hydroxybutyrate in the copolymer is greater than 80%, or 82%, or 85%, or 88%, or 90%, or 92%, preferably greater than 95% of the monomers in the copolymer.

In some embodiments, about 0.1 to 30 wt % of GBL product from the conversion reaction can be recycled back to the evaporator. In some embodiments, about 0.5 to 30 wt % of GBL product, or about 0.5 to 20 wt % of GBL product, or about 0.5 to 10 wt % of GBL product, or about 1 to 30 wt % of GBL product, or about 1 to 20 wt % of GBL product, or about 2 to 25 wt % of GBL product, or about 2 to 20 wt % of GBL product, or about 2 to 15 wt % of GBL product, or about 3 to 20 wt % of GBL product, or about 3 to 15 wt % of GBL product, or about 1 to 10 wt % of GBL product, or about 2 to 10 wt % of GBL product, is recycled back to the evaporator. Here, when vapor GBL is recycled, the recycling ratio can be converted to corresponding volume %.

In some embodiments, the final GBL is further processed for production of other desired commodity and specialty products, for example 1,4-butanediol (BDO), tetrahydrofuran (THF), N-methylpyrrolidone (NMP), N-ethylpyrrolidone (NEP), 2-pyrrolidinone, N-vinylpyrrolidone (NVP), polyvinylpyrrolidone (PVP) and the like.

As used herein, “biomass” or “starting biomass” is intended to mean any genetically engineered biomass from a host (e.g., recombinant bacteria) that includes a non-naturally occurring amount of the polyhydroxyalkanoate polymer e.g. poly-4-hydroxybutyrate (P4HB), as disclosed in U.S. Pat. No. 9,084,467, the content of which is incorporated herein by reference. The polyhydroxyalkanoate polymer comprises homopolymers of 4-hydroxybutyrate, copolymers of 4-hydroxybutyrate with other monomer such as 3-hydroxybutyrate, or mixtures thereof.

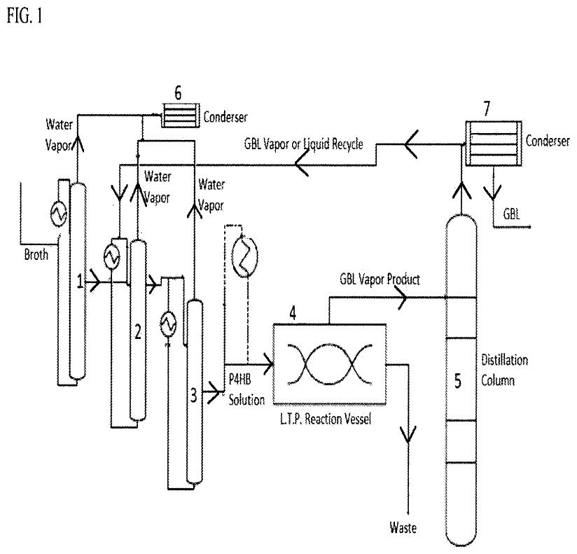

Referring to FIG. 1, an exemplary embodiment of the present disclosure includes at least a triple effect evaporator containing a first evaporator 1, a second evaporator 2, a third evaporator 3, a reaction vessel 4, a distillation column 5, a condenser 6 for collecting water vapor, and a condenser 7 for collecting GBL vapor. The first evaporator 1, the second evaporator 2, and the third evaporator 3 are in fluid communication with each other in serial in this order. Although three evaporators are illustrated in FIG. 1, the present disclosures are not limited thereto.

A fermentation broth (starting biomass) containing 10% to 50% by weight of biomass (based on the total weight of the broth) including P4HB is fed to the first evaporator 1 and is heated at about 60° C.-100° C. under vacuum or at atmospheric pressure by an apparatus known in the art for an amount of time to reduce water content. Water vapor is removed to a vessel such as a condenser 6. About 20% to 50%, or about 25% to 45%, or about 30% to 40%, by weight of the water based on the total weight of the broth are removed in the first evaporator.

The liquid with a lower water content is transferred to the second evaporator 2, where a recycled GBL stream is added replacing the water removed in the first evaporator. The second evaporator is heated at about 60° C.-100° C. under vacuum or at atmospheric pressure by an apparatus known in the art for an amount of time to further reduce water content. Water vapor is removed to a vessel such as a condenser 6. About 20% to 45%, or about 25% to 40%, or about 30% to 35%, by weight of the water based on the total weight of the broth are further removed in the second evaporator 2. As the GBL content increases toward the third evaporator 3, the temperature and pressure can be adjusted to facilitate efficient evaporation of remaining water.

The liquid biomass with further lower water content (i.e., a mixture of biomass, GBL, and remaining water) is transferred to the third evaporator 3, which is heated at about 60° C.-100° C. under vacuum or at atmospheric pressure by an apparatus known in the art for an amount of time to reduce water content. Remaining water from the broth is removed to a vessel such as a condenser 6, providing a slurry essentially containing biomass including P4HB (P4HB biomass) and GBL where the biomass is substantially free of water. The slurry can be directly fed to a reaction vessel 4 for combining with a catalyst. The heating temperature of the reaction vessel is generally lower than convention process by about 50° C.-130° C., or about 60° C.-120° C., or about 70° C.-115° C., or about 80° C.-110° C., or about 85° C.-105° C., or about 90° C.-100° C., or about 100° C. The reaction vessel may be LTP (low temperature pyroloysis) Unit Spray pyrolysis/Fluid Bed/or regular reaction vessel, etc. Or the slurry can be preheated to 300° C. and sent to LTP spray nozzle for flash pyrolysis. The slurry (unit liquid feed) has 3× better heat transfer than conventional dried biomass powder obtained by spray drying or drum drying.

In other embodiments, the slurry (which is also referred to as “substantially water-free biomass suspension or solution” or “biomass suspension or solution”) is optionally stirred, mixed, vortexed, or agitated to further promote extraction of P4HB from the host cells into GBL.

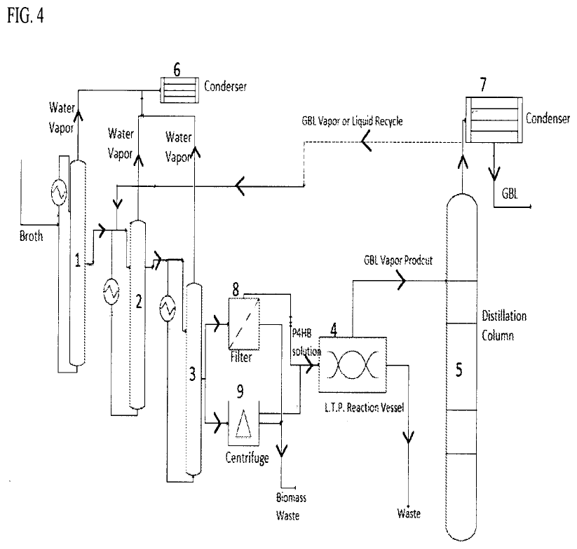

In another embodiments, the slurry is optionally treated by centrifugation, precipitation, or filtration to remove debris, as shown in FIG. 4, prior to be fed to the reaction vessel 4.

The term “substantially free of water” or “substantially water-free” as used herein is intended to refer that the biomass that is introduced to a conversion reaction (i.e., combining with catalyst) contains about 5 wt % or less, about 4.5 wt % or less, about 4 wt % or less, about 3.5 wt % or less, about 3 wt % or less, about 2.5 wt % or less, about 2 wt % or less, about 2.5 wt % or less, about 2 wt % or less, about 1.5 wt % or less, or about 1 wt % or less, based on the weight of the biomass suspension or solution that is subject to conversion reaction. The water content of the biomass suspension or solution (existing or flowing the system) can be measured by a method known in the art.

Even though in FIG. 1, a recycle stream of GBL is added in the second evaporator, the present disclosure is not limited thereto. The recycle stream of GBL can be added to other evaporators such as the third evaporator or to more than one evaporator. Similarly, the evaporator may comprise one or more, two or more, three or more, and the like, and the number of evaporator columns may be determined based on the amount of the starting biomass, water content of the starting biomass, energy efficiency, or a total cost of the process.

Combining P4HB Biomass Substantially Free of Water with Catalyst

According to embodiments, as the P4HB biomass substantially free of water is a suspension or solution due to the liquid GBL functioning as a solvent, no water is added to the conversion reaction.

In the reaction vessel 4, the P4HB biomass is combined with a catalyst under suitable conditions to help convert the P4HB polymer to high purity gamma-butyrolactone product. The catalyst (in solid or solution form) and biomass are combined for example by mixing, flocculation, centrifuging or spray drying, or other suitable method known in the art for promoting the interaction of the biomass and catalyst driving an efficient and specific conversion of P4HB to gamma-butyrolactone. Under “suitable conditions” refers to conditions that promote the catalytic reaction. For example, under conditions that maximize the generation of the product gamma-butyrolactone such as in the presence of co-agents or other material that contributes to the reaction efficiency. Other suitable conditions include in the absence of impurities, such as metals or other materials that would hinder the reaction from progression.

As used herein, “catalyst” refers to a substance that initiates or accelerates a chemical reaction without itself being affected or consumed in the reaction. Examples of useful catalysts include metal catalysts. In certain embodiments, the catalyst lowers the temperature for initiation of thermal decomposition and increases the rate of thermal decomposition at certain pyrolysis temperatures (e.g., about 190° C. to about 300° C.).

In some embodiments, the catalyst is a chloride, oxide, hydroxide, nitrate, phosphate, sulphonate, carbonate or stearate compound containing a metal ion. Examples of suitable metal ions include aluminum, antimony, barium, bismuth, cadmium, calcium, cerium, chromium, cobalt, copper, gallium, iron, lanthanum, lead, lithium, magnesium, molybdenum, nickel, palladium, potassium, silver, sodium, strontium, tin, tungsten, vanadium or zinc and the like. In some embodiments, the catalyst is an organic catalyst that is an amine, azide, enol, glycol, quaternary ammonium salt, phenoxide, cyanate, thiocyanate, dialkyl amide and alkyl thiolate. In some embodiments, the catalyst is calcium hydroxide. In other embodiments, the catalyst is sodium carbonate. Mixtures of two or more catalysts are also included.

In certain embodiments, the amount of metal catalyst is about 0.1% to about 15% or about 1% to about 25%, or 4% to about 50%, based on the weight of metal ion relative to the weight of the substantially water-free biomass. In some embodiments, the amount of catalyst is between about 7.5% and about 12%. In other embodiments, the amount of catalyst is about 0.5%, about 1%, about 2%, about 3%, about 4%, about 5%, about 6%, about 7%, about 8%, about 9%, or about 10%, or about 11%, or about 12%, or about 13%, or about 14%, or about 15%, or about 20%, or about 30%, or about 40%, or about 50%, or amounts in between these, relative to the weight of the substantially water-free biomass.

As used herein, the term “sufficient amount” when used in reference to a chemical reagent in a reaction is intended to mean a quantity of the reference reagent that can meet the demands of the chemical reaction and the desired purity of the product.

### Thermal Degradation of the P4HB Biomass

“Pyrolysis” and “thermolysis” as used herein refer to thermal degradation (e.g., decomposition) of the P4HB biomass for conversion to GBL. In general, the thermal degradation of the P4HB biomass occurs at an elevated temperature in the presence of a catalyst. For example, in certain embodiments, the heating temperature for the processes described herein is between about 190° C. to about 300° C. In some embodiments, the heating temperature is about 195° C. to about 275° C., or about 200° C. to about 260° C., or about 205° C. to about 250° C., or about 210° C. to about 225° C. In other embodiments, the heating temperature is about 190° C.-220° C. The heating temperature of the reaction vessel is generally lower than convention process by about 50° C.-130° C., or about 60° C.-120° C., or about 70° C.-115° C., or about 80° C.-110° C., or about 85° C.-105° C., or about 90° C.-100° C., or about 100° C.

“Pyrolysis” typically refers to a thermochemical decomposition of the biomass at elevated temperatures over a period of time. The duration can range from a few seconds to hours. In certain conditions, pyrolysis occurs in the absence of oxygen or in the presence of a limited amount of oxygen to avoid oxygenation. The processes for P4HB biomass pyrolysis can include direct heat transfer or indirect heat transfer. “Flash pyrolysis” refers to quickly heating the biomass at a high temperature for fast decomposition of the P4HB biomass, for example, depolymerization of a P4HB in the biomass. Another example of flash pyrolysis is RTP™ rapid thermal pyrolysis. RTP™ technology and equipment from ENVERGENT TECHNOLOGIES, Des Plaines, Ill. converts feedstocks into bio-oil.

In some embodiments, the heating is done in a vacuum, at atmospheric pressure or under controlled pressure. In certain embodiments, the heating is accomplished without the use or with a reduced use of petroleum generated energy.

In certain embodiments, the heating of the P4HB biomass/catalyst mixture is carried out for a sufficient time to efficiently and specifically convert the P4HB biomass to GBL. In certain embodiments, the time period for heating is from about 30 seconds to about 1 minute, from about 30 seconds to about 1.5 minutes, from about 1 minute to about 10 minutes, from about 1 minute to about 5 minutes or a time between, for example, about 1 minute, about 2 minutes, about 1.5 minutes, about 2.5 minutes, about 3.5 minutes.

In other embodiments, the time period is from about 1 minute to about 2 minutes. In still other embodiments, the heating time duration is for a time between about 5 minutes and about 30 minutes, between about 30 minutes and about 2 hours, or between about 2 hours and about 10 hours or for greater than 10 hours (e.g., 24 hours).

In certain embodiments, the heating temperature is at a temperature of about 190° C. to about 300° C. including a temperature in-between, for example, about 195° C., about 200° C., about 205° C., about 210° C., about 215° C., about 220° C., about 225° C., about 230° C., about 235° C., about 240° C., about 245° C., about 250° C., about 255° C. about 260° C., about 270° C., about 275° C., about 280° C., about 290° C. In certain embodiments, the temperature is about 200° C. In certain embodiments, the temperature is about 205° C., or about 210° C., or about 220° C., or about 230° C.

In certain embodiments, the process also includes flash pyrolyzing the residual biomass for example at a temperature of 400° C. or greater for a time period sufficient to decompose at least a portion of the residual biomass into pyrolysis liquids. In certain embodiments, the flash pyrolyzing is conducted at a temperature of about 400° C. to 750° C. or about 450° C. to 725° C., or about 475° C. to 700° C., or about 500° C. to 675° C., or about 525° C. to 650° C., or about 550° C. to 625° C. In some embodiments, a residence time of the residual biomass in the flash pyrolyzing is from 1 second to 15 seconds, or from 1 second to 5 seconds or for a sufficient time to pyrolyze the biomass to generate the desired pyrolysis precuts, for example, pyrolysis liquids.

As used herein, “pyrolysis liquids” are defined as a low viscosity fluid typically containing sugars, aldehydes, furans, ketones, alcohols, carboxylic acids and lignins. Also known as bio-oil, this material is produced by pyrolysis, typically fast pyrolysis of biomass at a temperature that is sufficient to decompose at least a portion of the biomass into recoverable gases and liquids that may solidify on standing. In some embodiments, the temperature that is sufficient to decompose the biomass is a temperature between about 400° C. to 800° C., or about 450° C. to 750° C., or about 500° C. to 700° C., or about 550° C. to 650° C.

In certain embodiments, “recovering” the gamma-butyrolactone vapor includes condensing the vapor. As used herein, the term “recovering” as it applies to the vapor means to isolate it from the P4HB biomass materials, for example including but not limited to: recovering by condensation, separation methodologies, such as the use of membranes, gas (e.g., vapor) phase separation, such as distillation, and the like. Thus, the recovering may be accomplished via a condensation mechanism that captures the monomer component vapor, condenses the monomer component vapor to a liquid form and transfers it away from the biomass materials.

As a non-limiting example, the condensing of the GBL vapor may be described as follows. The incoming gas/vapor stream from the pyrolysis chamber 4 enters a distillation column 5, where the gas/vapor stream may be pre-cooled. The gas/vapor stream then passes through a chiller where the temperature of the gas/vapor stream is lowered to that required to condense the designated vapors from the gas by indirect contact with a refrigerant. The gas and condensed vapors flow from the chiller into a condenser 7, where the condensed vapors are collected in the bottom. A part of the vapor from distillation column 5 or liquid from condenser 7 is recycled back to the evaporator as the recycle GBL stream. The gas, free of the vapors, flows from the condenser, and exits the unit. The recovered liquids flow, or are pumped, from the bottom of the condenser to storage. For some of the products, the condensed vapors solidify and the solid is collected.

In certain embodiments, recovery of the catalyst is further included in the processes of the disclosure. For example, when a calcium catalyst is used calcination is a useful recovery technique. Calcination is a thermal treatment process that is carried out on minerals, metals or ores to change the materials through decarboxylation, dehydration, devolatilization of organic matter, phase transformation or oxidation. The process is normally carried out in reactors such as hearth furnaces, shaft furnaces, rotary kilns or more recently fluidized beds reactors. The calcination temperature is chosen to be below the melting point of the substrate but above its decomposition or phase transition temperature. Often this is taken as the temperature at which the Gibbs free energy of reaction is equal to zero. For the decomposition of CaCO₃ to CaO, the calcination temperature at ΔG=0 is calculated to be −850° C. Typically for most minerals, the calcination temperature is in the range of about 800-1000° C. or about 850-950° C. but calcinations can also refer to heating carried out in the 200-800° C. range.

To recover the calcium catalyst from the biomass after recovery of the GBL, one would transfer the spent biomass residue directly from pyrolysis into a calcining reactor and continue heating the biomass residue in air to 825-850° C. for a period of time to remove all traces of the organic biomass. Once the organic biomass is removed, the catalyst could be used as is or purified further by separating the metal oxides present (from the fermentation media and catalyst) based on density using equipment known to those in the art.

In certain embodiments, the process can be selective for producing gamma-butyrolactone product with a smaller amount of undesired side products (e.g., dimerized product of GBL (3-(dihydro-2(3H)-furanylidene)dihydro-2(3H)-furanone), other oligomers of GBL or other side products). For example, it is possible to employ the use of a specific catalyst in a sufficient amount will reduce the production of undesired side products and increase the yield of GBL by at least about 2 fold. In some embodiments, the production of undesired side products will be reduced to at least about 50%, at least about 40%, at least about 30%, at least about 20% at least about 10%, or about at least 5%. In certain embodiment, the undesired side products will be less than about 5% of the recovered GBL, less than about 4% of the recovered GBL, less than about 3% of the recovered GBL, less than about 2% of the recovered GBL, or less than about 1% of the recovered GBL.

The processes described herein can provide a yield of GBL express as a percent yield, for example, when grown from glucose as a carbon source, the yield is up to 99%, or up to 98%, or up to 97%, or up to 96%, or up to 95% based gram GBL recovered per gram P4HB contained in the biomass feed to the process (reported as percent). In other embodiments, the yield is in a range between about 60% and about 95%, for example between about 65% and about 90%, or between about 70% and 90%. In other embodiment, the yield is about 98%, about 95%, about 92%, about 90%, about 88%, about 85%, about 80%, or about 75%.

As used herein, “gamma-butyrolactone” or GBL refers to the compound with the following chemical structure:

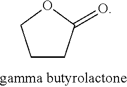

The term “gamma-butyrolactone product” or “GBL” refers to a product that contains at least about 80 up to 100 weight percent gamma-butyrolactone. For example, in a certain embodiment, the gamma-butyrolactone product may contain 95% by weight gamma-butyrolactone and 5% by weight side products. In some embodiments, the amount of gamma-butyrolactone in the gamma-butyrolactone product is about 81% by weight, about 82% by weight, about 83% by weight, about, 84% by weight, about 85% by weight, about 86% by weight, about 87% by weight, about 88% by weight, about 89% by weight, about 90% by weight, 91% by weight, about 92% by weight, about 93% by weight, about 94% by weight, about 95% by weight, about 96% by weight, about 97% by weight, about 98% by weight, about 99% by weight or about 100% by weight. In particular embodiments, the weight percent of gamma-butyrolactone product produced by the processes described herein is 85%, or greater than 85%, or greater than 90%, or greater than 95%.

In other embodiments, the gamma-butyrolactone product can be further purified if needed by additional methods known in the art, for example, by distillation, by reactive distillation (e.g., the gamma-butryolactone product is acidified first to oxidize certain components (e.g., for ease of separation) and then distilled) by treatment with activated carbon for removal of color and/or odor bodies, by ion exchange treatment, by liquid-liquid extraction-with GBL immiscible solvent (e.g., nonpolar solvents, like cyclopentane or hexane) to remove fatty acids etc., for purification after GBL recovery, by vacuum distillation, by extraction distillation or using similar methods that would result in further purifying the gamma-butyrolactone product to increase the yield of gamma-butyrolactone. Combinations of these treatments can also be utilized. Specifically, the gamma-butyrolactone product can be further purified by the methods disclosed in US 2014/0170714 A1, the content of which is incorporated herein by reference.

In certain embodiments, GBL is further chemically modified and/or substituted to other four carbon products (4C products) and derivatives including but not limited to succinic acid, 1,4-butanediamide, succinonitrile, succinamide, N-vinyl-2-pyrrolidone (NVP), 2-pyrrolidone (2-Py), N-methyl-2-pyrrolidone (NMP), tetrahydrofuran (THF), 1,4-butanediol (BDO). Methods and reactions for production of these derivatives from gamma-butyrolactone are readily known by one skilled in the art.

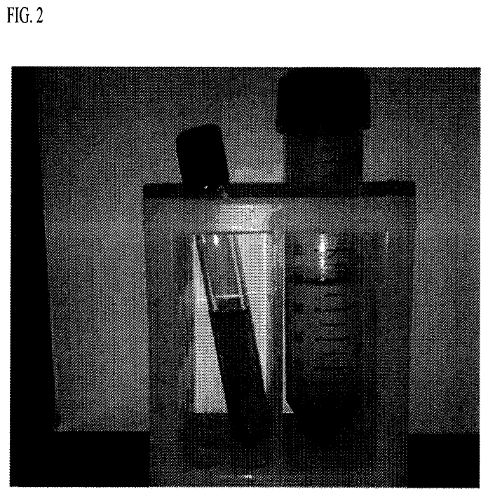

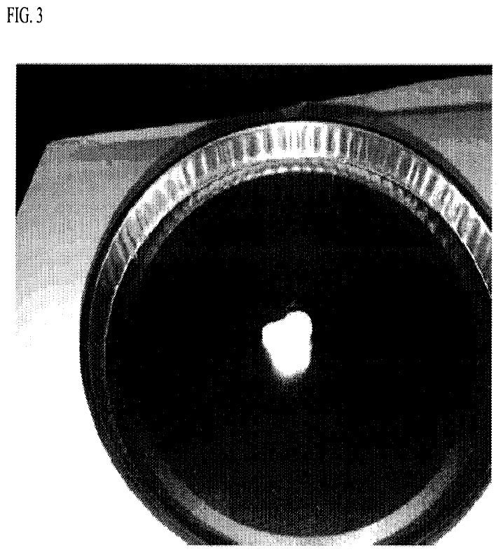

The inventors have demonstrated P4HB solutions as high as 30% @300,000 Daltons in the lab and have done an extraction by stripping out the water and confirmed that P4HB was in solution by crashing the solution into water resulting in a nice chunk of white polymer, as shown in FIGS. 2 and 3. The test tube on the left in FIG. 2 shows 30% solution of 300,000 Daltons P4HB in GBL. The test tube on the right in FIG. 2 shows extraction of P4HB from biomass with GBL in Rotovap. FIG. 3 shows P4HB extracted using GBL and precipitated into water.

An alternative example of this process is shown in FIG. 4. In addition to the evaporators 1, 2 and 3, the reaction vessel 4, the distillation column 5, the condenser 6 and the condenser 7, as shown in FIG. 1, a filter 8 or a centrifuge 9 is provided between the 3rd evaporator 3 and the reaction vessel 4. The same steps as described relative to the process shown in FIG. 1 are incorporated herein by reference. The filter 8 or the centrifuge 9 can be appropriately selected by one of ordinary skill in the art.

### EXAMPLES

The following examples are provided for illustrative purposes only and are in no way intended to limit the scope of the invention.

### Example 1. Production of Starting Biomass From Genetically Engineered Microbe Producing Poly-4-Hydroxybutyrate (P4HB)

Biomass containing poly-4-hydroxybutyrate (poly-4HB) was produced in a 20 L New Brunswick Scientific fermentor (BioFlo 4500) using a genetically modified E. coli strain specifically designed for high yield production of poly-4HB from glucose syrup as a carbon feed source. Examples of the E. coli strains, fermentation conditions, media and feed conditions are described in U.S. Pat. Nos. 6,316,262; 6,689,589; 7,081,357; and 7,229,804, the contents of which are incorporated herein by reference. The E. coli strain generated a fermentation broth which has a PHA titer of approximately 100-120 g of PHA/kg of broth. After fermentation, the broth was washed with deionized (DI) water by adding an equal volume of water, mixing for 2 minutes, centrifuging and decanting the water. The washed broth contained about 10% by weight of P4HB, 10% by weight of biomass and salts, and 80% by weight of water, based on the total weight of the broth.

### Example 2. Production of Gamma-Butyrlactone (GBL) From the Starting Biomass

Next, 1000g of the washed broth obtained in Example 1 was fed to the first evaporator and was heated at 100° C. for 30 mins to remove about 40% of water based on the total weight of the broth. The resulting solution was transferred to the second evaporator, where a recycled stream of GBL was fed to replace the water removed in the first evaporator. The mixture was heated at 100° C. for 30 mins to remove about 40% more of water based on the total weight of the broth. The resulting solution is transferred to the third evaporator and is heated at 100° C. for 20 mins to remove remaining water. The resulting slurry contained about 25% by weight of P4HB, about 25% by weight of biomass and salts, about 47% by weight of GBL, and less than 3% water, based on the total weight of the slurry.

While not required, the slurry (substantially water-free biomass solution/suspension) was subjected to filtration to even increase the purity of the GBL product. The filtration produced a polymer solution containing about 30% by weight of P4HB, about 3% by weight of biomass and salts, and about 66% by weight of GBL, based on the total weight of the polymer solution.

The resulting filtered substantially water-free biomass solution was mixed with lime (Ca(OH)₂ standard hydrated lime 98%, Mississippi Lime) targeting 4% by weight based on P4HB. Pyrolysis of the GLB+P4HB+Ca(OH)₂ was carried out using a flask equipped with a stirrer under vortex or stirring at 200-250° C. At the start of the process, a weighed sample of GLB+P4HB+Ca(OH)₂ was placed inside of the flask and a nitrogen purge flow established. The stirring and heat up were then started. As the temperature of the flask reaches its set point value (e.g., approximately 204° C.), gases generated by the GLB+P4HB+Ca(OH)₂ sample were swept out of the flask by the nitrogen purge and entered a series of glass condensers or chilled traps. The condensers consisted of a vertical, cooled glass condenser tower with a condensate collection bulb located at its base. A glycol/water mixture held at 0° C. was circulated through all of the glass condensers. The cooled gases that exited the top of the first condenser were directed downward through a second condenser and through a second condensate collection bulb before being bubbled through a glass impinger filled with deionized water.

Total pyrolysis run time was approximately 60 minutes.

After the completion of the pyrolysis run, the condensates from the condensers were collected and weighed. The results showed that the combined condensate weight was approximately 240 g. Analysis of the condensate by Karl Fisher moisture analysis and GC-MS showed that the condensate contained 3% water, 0.06% fatty acids with the balance of the material being GBL products. The GBL product yield ((g of GBL product/g of starting P4HB)×100%) therefore was calculated to be approximately 87%. Impurities such as GBL dimer, organo-sulfur and amide compounds were not detected as being present in the condensate by GC-MS, showing that the purity of the GBL product from the pyrolysis was about 99%.

Experiment was conducted to confirm that the process according to an embodiment employing recycle GBL to produce a substantially water-free biomass solution/suspension and subjecting it to a conversion process surprisingly effectively convert P4HB to GBL. Some sigma GBL was doped with a known amount of Undecane. Spray dried P4HB biomass was added to the sigma GBL and heated to 190° C. with reflux and was held there for 6 hours. Samples (GBL KS-1, KS-2, KS-3, and KS-4) were taken at reaction time 0, 30, 90, and 300 minutes and analyzed for determination of the GBL to Undecane ratio. Table 1 below shows that the ratio of Undecane to GBL (“ratio” column) and the Undecane level (PPM UD) decrease as time extends from time 0 to time 300 minutes.

TABLE 1
GBL reaction kinetics using GBL as carrier solvent

| | Temper | Time | GBL area | Undecane | | |
|---|---|---|---|---|---|---|
| Samples | ature | (min) | count | area count | ratio | PPM UD |
| GBL KS-1 | 25 | 0 | 567131264 | 1121739 | 0.20% | 1183.9045 |
| GBL KS-2 | 194 | 30 | 564413957 | 319808 | 0.06% | 337.53138 |
| GOL KS-3 | 194 | 90 | 472606060 | 193947 | 0.04% | 204.69531 |
| GBL KS-4 | 194 | 300 | 509453148 | 137114 | 0.03% | 144.7127 |

### Example 3. Post Purification of Biobased GBL by Distillation, Steam Stripping and Peroxide Treatment

This example outlines an optional procedure for the purification of biobased GBL liquid prepared from pyrolysis of a genetically engineered microbe producing poly-4-hydroxybutyrate polymer mixed with a catalyst as outlined above in Example 2. It should be noted that as the product from Example 2 is already very pure, this distillation purification process may not be required.

The GBL purification is a batch process whereby the “crude” GBL liquid recovered after pyrolysis is first filtered to remove any solid particulates (typically <1% of the total crude GBL weight) and then distilled twice to remove compounds contributing to odor and color.

Filtration of the crude GBL liquid is carried out on a lab scale using a Buchner fritted-glass funnel coupled to an Erlenmeyer receiving flask. Approximately 1 liter of crude GBL was filtered which resulted in approximately 0.99 liters of recovered GBL liquid.

The distillation of the filtered GBL liquid is carried out using a high vacuum 20 stage glass distillation column. The stage section of the column was contained inside a silver-coated, evacuated, glass insulating sleeve in order to minimize any heat losses from the column during the distillation process. The distillation is performed under vacuum conditions using a vacuum pump equipped with a liquid nitrogen cold trap. Typical column operating pressures during distillation are in the 25 in. Hg range. Cooling water, maintained at 10° C., is run through the condenser at the top of the column to assist in the fractionation of the vapor. The column is also fitted with two thermocouples: one at the top of the column to monitor vapor temperature and one at the bottom of the column to monitor the liquid feed temperature. At the start of the distillation, approximately 1 liter of filtered GBL liquid is charged into the bottom of the column, the condenser cooling water and the vacuum are then turned on. Once the pressure is stabilized, the filtered GBL liquid is slowly heated using a heating mantle to the boiling point of GBL (204° C.).

During the initial stages of the distillation, water contained in the filtered GBL is removed first and discarded along with lower boiling impurities. When the water and lower boiling impurities are completely removed, the GBL liquid feed temperature increases to the boiling point of GBL. At this stage, the vapor generated at the top of the column is mostly GBL which is condensed, collected and reserved for further distillation. When it is observed that the temperature of the liquid feed increased quickly above 204° C., the distillation is stopped. The total amount of GBL liquid recovered in the first distillation is 0.9 liters with a purity of 97%.

Another variation for the second distillation is tried whereby 1-3% (by weight GBL) of a 30% hydrogen peroxide solution is added along with the DI water to the previously distilled GBL liquid. The peroxide acts to oxidize the impurities in the GBL liquid making them less volatile and thereby easier to separate. To carry out this distillation, 0.9 liters of previously distilled GBL liquid is added to the bottom of the distillation column along with 203 g of DI water and 10.2 g of 30-32% hydrogen peroxide (Sigma Aldrich). The condenser cooling water and vacuum are started and the GBL liquid feed heated. The distillation generated a water fraction first and second transitional fraction prior to the pure GBL vapor. Both the first and second fractions are discarded and the pure GBL liquid collected. Analysis of the GBL liquid by GC-MS showed that is >99.5% pure with very low odor and color. To remove additional water, the purified GBL liquid can be stored over dry molecular sieves (3-4 Å pore size, Sigma Aldrich) until used. A part of thus purified GBL liquid is recycled back to evaporator of Example 2.

The following Table 2 is a summary of the heat balance on the existing process including drying of starting biomass where most of the water from fermentation is evaporated using a spray drier (“base case process”) compared to the process of Example 2. As can be seen from Table 1, as the recycle ratio (GBL recycled/product GBL) increases, the required evaporator surface area reduces and the heat from the recycled GBL can be efficiently used for evaporation.

Summary of Energy Used by Base Case Process and GBL Recycle Process

| | | | Approximate | | | | | | |
|---|---|---|---|---|---|---|---|---|---|
| | Flow | | U value | | | | | | |
| | Rate of | Solids | necessary | | | | | | Approx |
| | GAL to | Concentration | to be able | | Total | Heat from | | | imate |
| Recycle Ratio | first | entering | to use 2 | Heat Duty | Heat | GBL/Heat | Evaporator | Steam Required | Diameter |
| (GBL | distillation | Thermolysis | Bepex | on | from | needed | Surface | for | of GBL |
| recycled/product | column | Unit (out of | paddle | Thermolysis | Recycled | for | Area | Evaporation/ | Vacuum |
| GBL) | (kg/hx) | evaporators) | dryers | Unit | GBL | Evaporation | Required | Drying | Column |
| lb/hr/lb/hr | kg/hr | (%w/w) | BTU/hr-ft [symbol] F | kW | kW | (%kW/kW) | m² | kg/hr | m |
| Base case | 3,464 | 98% | 15 | 1,946 | N/A | 0% | 338 | 30,724 | 0.75 |
| process Spray | | | | | | | | | |
| drying biomass | | | | | | | | | |
| 1 | 6,764 | 65% | 48 | 3,206 | 766 | 5.2% | 318 | 5,377 | 1.05 |
| 2 | 9,964 | 49% | 62 | 4,427 | 1,480 | 11.0% | 301 | 5,075 | 1.29 |
| 3 | 13,298 | 39% | 86 | 5,700 | 2,224 | 16.5% | 282 | 4,761 | 1.49 |
| 4 | 16,564 | 32.5% | 105 | 6,947 | 2,953 | 21.9% | 264 | 4,453 | 1.67 |
| 5 | 20,128 | 27.5% | 125 | 8,308 | 3,748 | 27.8% | 244 | 4,118 | 1.83 |

[symbol]

indicates data missing or illegible when filed

Another variation on the above purification steps is to add DI water and/or 30% hydrogen peroxide solution during the first distillation stage. Additional purification steps could include treatment with ozone, ion exchange resin or activated carbon.

In another embodiment, it is also possible to subject the gamma-butyrolactone generated from processes described herein directly to hydrogenation, esterification or amidation conditions to produce the corresponding diol, hydroxyl ester and amide (e.g., 1,4-butanediol, alkyl 4-hydroxy butyrate, or N-alkyl 2-pyrrolidone when subjected to hydrogenation with H₂, esterification with alkyl alcohol and amidation with alkyl amine respectively).

The processing of fats and oils to produce alcohols provides some guidance in this respect. Oils and fats are significant sources of fatty alcohols that are used in a variety of applications such as lubricants and surfactants. The fats are not typically hydrogenated directly as the intensive reaction conditions tend to downgrade the glycerol to lower alcohols such as propylene glycol and propanol during the course of the hydrogenation. For this reason it is more conventional to first hydrolyze the oil and then pre-purify the fatty acids to enable a more efficient hydrogenation (see for instance Lurgi's hydrogenation process in Bailey's Industrial Oil and Fat Products, Sixth Edition, Six Volume Set. Edited by Fereidoon Shahidi, John Wiley & Sons, Inc. 2005).

While the subject matter disclosed herein has been described in connection with what is presently considered to be practical example embodiments, it is to be understood that the present disclosure is not limited to the disclosed embodiments, and covers various modifications and equivalent arrangements included within the spirit and scope of the appended claims.

Other than in the examples herein, or unless otherwise expressly specified, all of the numerical ranges, amounts, values and percentages, such as those for amounts of materials, elemental contents, times and temperatures of reaction, ratios of amounts, and others, in the above portion of the specification and attached claims may be read as if prefaced by the word “about” even though the term “about” may not expressly appear with the value, amount, or range.

Accordingly, unless indicated to the contrary, the numerical parameters set forth in the above specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains error necessarily resulting from the standard deviation found in its underlying respective testing measurements. Furthermore, when numerical ranges are set forth herein, these ranges are inclusive of the recited range end points (i.e., end points may be used). When percentages by weight are used herein, the numerical values reported are relative to the total weight.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10. The terms “one,” “a,” or “an” as used herein are intended to include “at least one” or “one or more,” unless otherwise indicated.

What is claimed is:

1. A method of producing gamma-butyrolactone (GBL) product from a starting biomass containing water and poly-4-hydroxybutyrate (P4HB)-containing cells and water, the method comprising:
(a) introducing the starting biomass to an evaporator,
(b) introducing liquid or vapor GBL to the evaporator as a solvent in a weight equivalent to or less than a weight of water removed to obtain a biomass suspension or solution that is substantially free of water and comprises P4HB dissolved or suspended in the GBL,
(c) combining the biomass suspension or solution that is substantially free of water and a conversion catalyst and heating resulting mixture to convert the P4HB to GBL, and
(d) collecting the GBL,
wherein the liquid or vapor GBL introduced in step (b) is a recycle GBL that is a part of the collected GBL obtained in step (d) and recycled back to the evaporator to be mixed as a solvent with the biomass, and
wherein the method does not include a drying.

2. The method of claim 1, which further comprises step (e) mixing, stirring, vortex, or agitation to promote extraction of P4HB from host cells into GBL.

3. The method of claim 1, which further comprises, prior to step (c), step (f) removing solids from the biomass suspension or solution that is substantially free of water, by filtration, precipitation, or centrifugation.

4. The method of claim 1, wherein no water is added in steps (a), (b), and (c).

5. The method of claim 1, wherein the evaporator comprises multiple serial evaporators that are in fluid communication with each other and the recycle GBL is introduced to one or more of the evaporators.

6. The method of claim 1, wherein the evaporator contains a first evaporator, a second evaporator, and a third evaporator, which are in fluid communication with each other in serial in this order, and the recycle GBL is introduced into the second evaporator and/or the third evaporator.

7. The method of claim 1, wherein the step (a) is carried out at a temperature of from about 60° C.-100° C. under vacuum or at atmospheric pressure .

8. The method of claim 6, wherein about 20-50% of water contained in the starting biomass is removed in the first evaporator, about 20-45% of water contained in the starting biomass is removed in the second evaporator, and about 5-35% water contained in the starting biomass is removed in the third evaporator; and wherein the recycled GBL is introduced to replace the water removed from the first and the second evaporator.

9. The method of claim 1, wherein the biomass solution or suspension that is substantially free of water has a water content of about 5 wt % or less based on the weight of the biomass solution or suspension.

10. The method of claim 1, wherein the step (a) is carried out at a temperature from about 70° C.-90° C. under vacuum.

11. The method of claim 1, wherein the step (a) is carried out for a time period from about 5 minutes to about 2 hours.

12. The method of claim 1, wherein the collected GBL in step (d) comprises less than 5% by weight of side products.

13. The method of claim 1, wherein the conversion of step (c) is carried out in the presence of a catalyst.

14. The method of claim 13, wherein the catalyst is sodium carbonate or calcium hydroxide.

15. The method of claim 1, wherein a yield of the GBL product is about 85% by weight or greater based on one gram of a GBL in a conversion product per gram of P4HB.

16. The method of claim 1, wherein the starting biomass is a genetically engineered biomass from a host cell that includes a non-naturally occurring amount of P4HB.

17. A biobased gamma-butyrolactone product produced by the method of claim 1.

18. The biobased gamma-butyrolactone product of claim 17, wherein the gamma-butyrolactone product comprises less than 5% by weight of side products.
